SEQUENCE LISTING (7 sequences)

SEQ ID NO: 1 - DNA, 1796 bp, Homo sapiens
  CDS (100)..(1263)
agggcgggggtcggcggcccggccagcccggcccggcccggggccgcgtcctgagagtcagccctcgccgctgcagcctcggcgcccggccggccggccatggagcgccccccgccccgcgccgccggccgggaccccagtgcgctgcgggccgaggcgccgtggctgcgcgcggagggtccggggccgcgcgccgcgcccgtgacggtgcccacgccgccgcagggctcttccgtgggcggcggcttcgcgggcttggagttcgcgcggccgcaggagtcggagccgcgggcctcggacctgggggccccccggacgtggacgggggcggcggcggggccccggactccgtcggcgcacatccccgtcccagcgcagagagccaccccaggaaaagcccggctggacgaggtcatggctgccgctgcccttacaagcctgtccaccagccctctccttctgggggccccagttgcagccttcagcccagagcctggcctggagccctggaaggaggccctggtgcggcccccaggcagctacagcagcagcagcaacagtggagactggggatgggacctggtcagtgaccagtcctctccgtccaccccgtcacccccactgccccccgaggcagcccactttctgtttggggagcccaccctgagaaaaaggaagagcccggcccaggtcatgttccagtgtctgtggaagagctgcgggaaggtgctgagcacggcgtcggcgatgcagagacacatccgcctggtgcacctggggaggcaggcagagcctgatcagagtgatggtgaggaggacttctactacacagagctggatgttggtgtggacacgctgaccgacgggctgtccagcctgactccagtgtcccccacggcctccatgccgcctgccttcccccgcctggagctgccagagctgctggagcccccagccctgcctagtcccctgcggccgcctgccccgcccctgcccccgccccctgtcctgagcaccgttgctaacccccagtcctgtcacagtgaccgtgtctaccagggctgcctgacgcccgcccgcctggagccgcagcccacggaggtcggagcctgcccacccgccttgtcctccaggatcggagtcaccctgaggaagccccgcggcgacgcgaagaagtgccggaaggtgtatggcatggagcgccgggacctctggtgcacagcctgccgctggaagaaagcctgccagcggttcctggactaagtccggctcgttcaagaacataagctaccaccttctccctccccaccccctccaggcccggggctgaaacagcccgaggacagccccaggggctggccttcaccagctgcagggtctgcttttacttggggtgggggggcggggctgaccctgaaccctcccccccgccaggtcggggaggggtcccaccactcaaagtgcctctaaagaaaccagctttttgcactaaagccaaaccacaccgctgtccccttagccccaagggccctgggggcagccaccctcccgcctgtcggcccgtagatttatcaagggtgttatgggcccagctttggggggccagtcccgatgcactttgaggggtgttggagaggggactcccccactcgcacttaactcaacggctctcgggccctggggctgtttttaccatgtttgtttttgaagctcaggtgtctcacgtctgggctgcaccaggcgaagagagaaattaaagatttgaggtttttccagaaaaaaaaaaaaaaaaaaaa

SEQ ID NO: 2 - protein, 387 aa, Homo sapiens [sequence omitted]

SEQ ID NO: 3 - DNA, 1874 bp, Homo sapiens
  CDS (133)..(1674)
aggttcaagtgcgcatgtgcgcgaggagtcgctcgggcacttattgagcgccgactgtctacgggcggccgggggtgatgggcagaggcttcagtgtccccttcgcctccgcaggagaggagaggcagcagcatggcgagtgtcctgtcccgacgccttggaaagcggtccctcctgggagcccgggtgttgggacccagtgcctcggaggggccctcggctgccccaccctcggagccactgctagaaggggccgctccccagcctttcaccacctctgatgacaccccctgccaggagcagcccaaggaagtccttaaggctcccagcacctcgggccttcagcaggtggcctttcagcctgggcagaaggtttatgtgtggtacgggggtcaagagtgcacaggactggtggagcagcacagctggatggagggtcaggtgaccgtctggctgctggagcagaagctgcaggtctgctgcagggtggaggaggtgtggctggcagagctgcagggcccctgtccccaggcaccacccctggagcccagagcccaggccctggcctacaggcccgtctccaggaacatcgatgtcccaaagaggaagtcggacgcagtggaaatggatgagatgatggcggccatggtgctgacgtccctgtcctgcagccctgttgtacagagtcctcccgggaccgaggccaacttctctgcttcccgtgcggcctgcgacccatggaaggagagtggtgacatctcggacagcggcagcagcactaccagcggtcactggagtgggagcagtggtgtctccaccccctcgcccccccacccccaggccagccccaagtatttgggggatgcttttggttctccccaaactgatcatggctttgagaccgatcctgaccctttcctgctggacgaaccagctccacgaaaaagaaagaactctgtgaaggtgatgtacaagtgcctgtggccaaactgtggcaaagttctgcgctccattgtgggcatcaaacgacacgtcaaagccctccatctgggggacacagtggactctgatcagttcaagcgggaggaggatttctactacacagaggtgcagctgaaggaggaatctgctgctgctgctgctgctgctgccgcaggcaccccagtccctgggactcccacctccgagccagctcccacccccagcatgactggcctgcctctgtctgctcttccaccacctctgcacaaagcccagtcctccggcccagaacatcctggcccggagtcctccctgccctcaggggctctcagcaagtcagctcctgggtccttctggcacattcaggcagatcatgcataccaggctctgccatccttccagatcccagtctcaccacacatctacaccagtgtcagctgggctgctgccccctccgccgcctgctctctctctccggtccggagccggtcgctaagcttcagcgagccccagcagccagcacctgcgatgaaatctcatctgatcgtcacttctccaccccgggcccagagtggtgccaggaaagcccgaggggaggctaagaagtgccgcaaggtgtatggcatcgagcaccgggaccagtggtgcacggcctgccggtggaagaaggcctgccagcgctttctggactgagctgtgctgcaggttctactctgttcctggccctgccggcagccactgacaagaggccagtgtgtcaccagccctcagcagaaaccgaaagagaaagaacggaaacacggagtttgggctctgttggctaaggtgtaacacttaaagcaattttctcccattgtgcgaacattttattttttaaaaaaaaaaaaaaaaaa

SEQ ID NO: 4 - protein, 513 aa, Homo sapiens [sequence omitted]

SEQ ID NO: 5 - DNA, 25 bp, Artificial sequence
  442:<2231> Description of Artificial sequence: Synthesized oligonucleotide
acccccgtaccacacataaaccttc

SEQ ID NO: 6 - DNA, 22 bp, Artificial sequence
  455:<2231> Description of Artificial sequence: Synthesized oligonucleotide
ggtgctgggagccttaaggact

SEQ ID NO: 7 - DNA, 530 bp, Homo sapiens
  ref <308>: DATABASE ACCESSION NUMBER: GenBank/L34020
  ref <309>: DATABASE ENTRY DATE: 1996-03-01
gttgagccccgcgccttcgcccgggtggggcgctgcgctgtcagcggcttgctgtgtgaggcagaacctgcgggggcaggggcgggctggttccctggccagccattggcagagtccgcaggctagggctgtcaatcatgctggccggcgtggccccgcctccgccggcgcggccccgcctccgccggcgcacgtctgggacgcaaggcgccgtgggggctgccgggacgggtccaagatggacggccgctcaggttctgcttttacctgcggcccagagccccattcattgccccggtgctgagcggcgccgcgagtcggcccgaggcctccggggactgccgtgccgggcgggagaccgccatggcgaccctggaaaagctgatgaaggccttcgagtccctcaagtccttccagcagcagcagcagcagcagcagcagcagcagcagcagcagcagcagcagcagcagcagcagcaacagccgccaccgccgccgccgccgccgccgcctcctcagcttcctcagcc

(19) United States
(12) Patent Application Publication
(10) Pub. No.: US 2005/0053940 A1
(43) Pub. Date: Mar. 10, 2005

(54) Huntington's disease gene transcriptional factors

(76) Inventors: Joh-e Ikeda, Tokyo (JP); Kazunori Tanaka, Kanagawa (JP)

Correspondence Address: WENDEROTH, LIND & PONACK, L.L.P., 2033 K STREET N. W., SUITE 800, WASHINGTON, DC, 20006-1021, US

(21) Appl. No.: 10/496,129

(22) PCT Filed: Nov. 21, 2002
(86) PCT No.: PCT/JP02/12178

(30) Foreign Application Priority Data
Nov. 21, 2001 (JP) 2001-356080

(51) Int. Cl. C12Q 1/68; C07H 21/04; C12N 9/22

(52) U.S. Cl. 435/6; 435/69.1; 435/320.1; 435/199; 435/325; 536/23.2

(57) ABSTRACT

The present invention provides an isolated and purified transcriptional factors for the Huntington's disease gene, which have the amino acid sequence of SEQ ID No. 2 or SEQ ID No. 4 as a protein factor associated with the transcription of the Huntington's disease gene product. The invention also provides a polynucleotides encoding such transcriptional factors. These inventions are useful in the development of a therapeutic technology for an artificial control of the production of a pathogenically mutated huntingtin in HD, or in the development of a therapeutic method or agent against a selective neuronal cell death in HD.

### TECHNICAL FIELD

The present invention relates to transcriptional factors for the Huntington's disease gene. More particularly, the invention relates to the transcriptional factors for the Huntington's disease gene, which are involved in a transcription of a gene product of the Huntington's disease gene, and which play an important role for example in the onset of Huntington's disease; and the present invention further relates to various genetic materials for utilizing these factors.

### BACKGROUND ART

Huntington's disease (HD) is one of neurodegenerative diseases exhibiting autosomal dominant inheritance. The causative essence of the HD onset is considered to be attributable to the formation of a polyglutamine sequence as a result of an abnormal expansion of the glutamine residue at the N-terminal of a protein huntingtin whose function is unknown and which is encoded in the HD pathogenic gene (HD gene); said extension is occurred corresponding to a specific increase in the CAG repeat sequence which is present within exon1 of the HD gene. The HD gene is also known to exhibit a selective neuronal degeneration and neuronal loss in spite of expressing concurrently in various tissues.

As an important cause for a selectivity of the neuronal cell death, a regulation of the expression of the causative gene is contemplated. In fact, the study of the mouse model suggests that a suppression of the expression of mutated huntingtin may improve the symptom of the disease (Cell, 101:57-66, 2000).

As described above, an elucidation of the mechanism of the HD gene expression is important and essential in the development of a novel therapeutic method or agent against the HD. However, no molecule associated with the transcription of the human HD gene (transcriptional factor) has been identified.

The present invention was established in view of the circumstance described above, and its objective is to provide a transcriptional factor for human HD gene.

Another objective of the invention is to provide polynucleotide encoded an HD transcriptional factor, antibody and the like.

### DISCLOSURE OF THE INVENTION

The first aspect of the invention is an isolated and purified transcriptional factor for the Huntington's disease gene, which has the amino acid sequence of SEQ ID No. 2 or SEQ ID No. 4. In this first aspect, the transcriptional factor for the Huntington's disease gene recognizes at least one of the 144th to 150th sequence, the 164th to 170th sequence and the 184th to 190th sequence that reside in transcription regulatory region of the Huntington's disease gene represented by the base sequence of SEQ ID No. 7.

The second aspect of the invention is an isolated and purified polynucleotide encoding each of the transcriptional factors for the Huntington's disease gene, described above.

This second aspect is typically a polynucleotide having the base sequence of SEQ ID No. 1 or SEQ ID No. 3.

The third aspect of the invention is an oligonucleotide consisting of a base sequence of 10 base pairs or more, which hybridizes with the polynucleotide described above.

The fourth aspect of the invention is a recombinant vector holding the polynucleotide described above.

The fifth aspect of the invention is transformed cell with the recombinant vector described above.

The sixth aspect of the invention is an antibody against the transcriptional factor Huntington's disease gene described above.

In the invention, the term “polynucleotide” or “oligonucleotide” does not mean a fragment having any specific number of the bases, and in a general rule here a fragment of 100 bp or more may be referred to as a polynucleotide and a fragment less than 100 bp may be referred to as an oligonucleotide, although there are some exceptions.

### BRIEF DESCRIPTION OF THE DRAWINGS

FIG. 1 shows a schematic representation of the structure of the HD gene used for One-Hybrid system to screen cDNA clones encoded an HD gene transcriptional factor, and regions corresponding to the probe DNAs.

FIG. 2 shows the results of the binding activity examination of the three clones, which were isolated by the One-Hybrid system, using strains containing each reporter construct.

FIG. 3 shows the results of the northern blott analysis examined an expression of the clone 2L mRNA and the clone 8 mRNA in a human tissue. Descriptions are as follows; Lane 1: Heart, 2: Brain, 3: Liver, 4: Pancreas, 5: Placenta, 6: Lung, 7: Stomach, 8: Jejunum, 9: Ileum, 10: Large intestine, 11: Rectum, 12: Muscle, 13: Uterus, 14: Urinary bladder, 15: Kidney, 16: Spleen, 17: Uterine cervix, 18: Ovary, 19: Testis, 20: Prostate.

FIG. 4A shows the results of a gel shift assay examined the binding activity of the clone 2L gene product with an HD gene fragment, and B shows the results of a gel shift assay that was investigated for a competitive activity.

FIG. 5A shows the results of a gel shift assay examined the binding activity of the clone 8 gene product with an HD gene fragment, and B shows the results of a gel shift assay that was investigated for a competitive activity.

FIG. 6 shows the results of a gel shift assay examined the binding region of the clone 2L gene product to the HD gene (right, electrophoresis image), and a schematic representation of the GST fusion protein used for this test (left).

FIG. 7 shows the results of DNase I footprint analysis examined the DNA sequence, which resides in an HD gene transcription regulatory region, recognized by the both products that are the C-terminal regions of both clone 2L and clone 8 gene products (left: whole electrophoresis image, right: magnified image), as well as a schematic representation of the position of the DNA fragment and the protected core sequence in the HD gene transcriptional regulatory region used for this test.

### BEST MODE FOR CARRYING OUT THE INVENTION

The first aspect of the invention is the HD gene transcriptional factors having the amino acid sequences represented by each of SEQ ID No. 2 and SEQ ID No. 4, hereinafter sometimes referred to as “HD/TF”. Concretely, it is the HD gene transcriptional factor having the amino acid sequence represented by SEQ ID No. 2 (HD/TF-1) and the HD gene transcriptional factor having the amino acid sequence represented by SEQ ID No. 4 (HD/TF-2).

Each of the transcriptional factor HD/TF-1 and HD/TF-2 according to the present invention is a protein that has at least one function in the function involved in the initiation of the HD gene transcription (transcriptional initiation factor), the function that up- or down-regulate the elongation of the transcription product (transcriptional elongation factor), and the function involved in the transcriptional regulation (transcriptional regulation factor). The HD gene has been known (Cell 72: 971-983, 1993; Genomics 25:707-715, 1995; GenBank Accession No. L34020). SEQ ID No. 7 is a partial genome sequence of this known HD gene. In the HD gene, the transcription is initiated from the 219th g or the 229th c in SEQ ID No. 7, and the protein is coded from the atg codon in the 364th to 366th positions. The transcriptional factors HD/TF-1 and HD/TF-2 according to the present invention recognize the “gccggcg” sequence which resides in the 144th to 150th sequence, the 164th to 170th sequence and the 184th to 190th sequence in the HD gene transcriptional regulatory region (thus, such inventive transcriptional factor binds to the “gccggcg”). Furthermore, these three “gccggcg” sequences has the characterization that each is successively located via a 13-base sequence. Accordingly, the inventive transcriptional factors HD/TF-1 and HD/TF-2 can be also defined as the proteins which bound to this specific “gccggcg” sequence.

The HD gene transcription factor according to the present invention is useful in the development of a therapeutic technology for an artificial regulation of the production of a pathogenically mutated huntingtin in HD or in the development of a therapeutic method or agent against a selective neuronal cell death in HD. Concretely, by the screening of a low molecular weight compound which has an antagonistic or promotive effect on this transcriptional factor, a novel therapeutic agent is expected to be developed. On the other hand, such a transcriptional factor or its partial fragment (peptide) can be used as an antigen for production of an antibody. For the sake of screening for the low molecular weight compound, it is also possible to utilize a sequence containing the gccggcg sequence described above (for example, a purified polynucleotide or purified oligonucleotide).

The transcriptional factors HD/TF-1 and HD/TF-2 according to the present invention can be respectively obtained by the isolation using human cells or by the preparation of a polypeptide via a chemical synthesis based on the amino acid sequence represented by each of SEQ ID Nos. 2 and 4. It is also possible to accomplish a large scale production by isolation and purification from the transformed cell according to the present invention. More particularly, the transformed cells is incubated; subsequently for example, the HD/TF-1 and the HD/TF-2 may respectively be collected from the culture in a large amount by following method: a denaturing agent such as urea or detergent, or an ultrasonic treatment, enzyme digestion, salting-out, solvent precipitation, dialysis, centrifugation, ultrafiltration, gel filtration, SDS-PAGE, isoelectric focusing, ion exchange chromatography, hydrophobic chromatography, affinity chromatography, reverse phase chromatography and the like. The HD/TF-1 and the HD/TF-2 obtained by such a gene engineering method include respective fusion proteins with any other proteins. For example, a fusion protein with glutathion-S-transferase (GST) or green fluorescent protein (GFP) can be mentioned. It is also possible that a protein expressed in cells is modified at various pathways in the cells after the translation. Accordingly, such a modified protein is also encompassed by the HD gene transcription factor. Such a posttranslational modification includes an N-terminal methionine cleavage, N-terminal acetylation, saccharide chain addition, intracellular protease limiting cleavage, myristoylation, isoprenylation, phosphorylation and the like.

The transcriptional factor according to the first aspect of the present invention includes a peptide consisting of a partial consecutive sequence in SEQ ID Nos. 2 and 4 (5 amino acid residues or more). Such a peptide, for example, can be used as an antigen for production of an antibody against the transcriptional factor according to the present invention.

The second aspect of the invention is a polynucleotide encoding the respective HD gene transcriptional factors described above. Such a polynucleotide includes a genome gene encoding each of the HD/TF-1 and the HD/TF-2, RNAs (mRNAs; SEQ ID Nos. 1 and 3) transcribed from genome gene and cDNAs synthesized from mRNAs. The polynucleotides represented by SEQ ID Nos. 1 and 3 may have a single-stranded form, a complementary strand thereof, and their double-stranded form.

The DNA of genome gene encoding each of the HD/TF-1 and the HD/TF-2, for example, can be isolated by screening a human genome DNA library using a polynucleotide or oligonucleotide probe consisting of the base sequence represented by each of SEQ ID Nos. 1 and 3 or a partial sequence thereof. The resultant genome gene can be amplified by an ordinary gene amplification method such as PCR (polymerase chain reaction) method, NASBN (nucleic acid sequence based amplification) method, TMA (transcription-mediated amplification) method and SDA (strand displacement amplification) method.

The cDNA can be also synthesized by using a poly (A)+ RNA extracted from human cells as a template. Such human cells may be one isolated from a human body by a surgery or may be culture cells. The cDNA can be synthesized by the conventional methods (Mol. Cell Biol. 2, 161-170, 1982; J. Gene 25, 263-269, 1983; Gene, 150, 243-250, 1994). Alternatively, for the sake of synthesizing the intended cDNA by RT-PCR method, an oligonucleotide can be used as a primer together with an mRNA template isolated from human cells. Such a cDNA, for example, can be used for a gene engineering production of the HD/TF-1 and the HD/TF-2.

The third aspect of the present invention is an oligonucleotide consisting of a base sequence of 10 base pairs or more, which hybridizes with the polynucleotide of the second aspect described above. Such an oligonucleotide is concretely an oligonucleotide probe used for the cloning of the polynucleotide, and an oligonucleotide primer used for PCR-amplification of the polynucleotide or the like.

The oligonucleotide probe is a DNA fragment or RNA fragment which hybridizes under a stringent condition with a genome gene described above or with a polynucleotide described above. For example, a DNA comprising consecutive 10 to 99 bases in the base sequence represented by SEQ ID No. 1 or 3 may be contemplated therein. As used herein, the stringent condition is a condition enabling a hybrid formation specifically with a gene or polynucleotide probe described above, and is defined on the basis of a concentration of salts and an organic solvents (such as formamide), and temperature in the steps of hybridization and washing. Typically, the details are specified in U.S. Pat. No. 6,100,037.

The oligonucleotide primer is at least a set of two oligonucleotides for PCR-amplification of a gene or polynucleotide described above. Such a primer set based on the base sequence of SEQ ID Nos. 1 or 3 can be designed and prepared through the steps of synthesis and purification. The primer design should take the followings into account. The primer size (number of bases) is 15 to 40 bases, preferably 15 to 30 bases in consideration of satisfying specifically annealing with a template DNA. However, at least 30 bases are effective when conducting an LA (long accurate) PCR. For avoiding any annealing between a pair of primers (two primers) consisting of a sense strand (5′ terminal) and an antisense strand (3′ terminal), the primer having complementary sequences between both primers should not be adopted. In addition, a primer having an self-complementary sequence should be also avoided in order to prevent the hairpin structure formation in respective the primers. Furthermore, the GC content should be about 50% to ensure a stable binding between the primer and template DNA, and the GC-rich or AT-rich region should not be distributed unevenly within the primer. Since the annealing temperature is dependent on the Tm (melting temperature), the primers whose Tms are close to each other within the range from 55 to 65° C. should be selected to obtain a highly specific PCR product. It should be also noted that the final concentration of a primer in PCR should be about 0.1 to about 1 μM. A commercial primer designing software such as Oligo™ [National Bioscience Inc., USA], GENETYX [SOFTWARE KAIHATSU, JAPAN] can be used.

The fourth aspect of the invention is a recombinant vector carrying the polynucleotide of the second aspect described above. Such a vector is a cloning vector or an expression vector, and may be selected appropriately on the basis of the type of a polynucleotide as an insert or the purpose of use. For example, when a cDNA or its ORF region is used as an insert to produce the HD/TF-1 or the HD/TF-2 in a gene engineering manner, expression vectors used for in vitro transcription or expression vectors, which are suitable for each of prokaryotes such as E. coli or Bacillus subtilis as well as eukaryotes such as yeast, insect cell, mammalian animal cell and the like, can be used. Also, when using a genome gene DNA as an insert, a BAC (bacterial artificial chromosome) vector or cosmid vector may be used.

The fifth aspect of the invention is transformed cells using the recombinant vector described above. For example, when the recombinant vector described above is used to produce the HD/TF-1 or the HD/TF-2 in a gene engineering manner, each of prokaryotes such as E. coli or Bacillus subtilis and eukaryotes such as yeast, insect cell, mammalian animal cell and the like can be used. The transformed cells can be prepared by incorporating the recombinant vector into the cells by means of a known method such as electroporation, calcium phosphate method, liposome method, DEAE dextran method and the like.

The sixth aspect of the invention is an antibody against the HD gene transcriptional factor described above. This antibody is a polyclonal antibody or a monoclonal antibody which recognizes the HD gene transcriptional factor, and includes a whole molecule, which is able to bind to respective epitopes of the HD/TF-1 and the HD/TF-2, as well as Fab, F(ab′)₂, Fv fragments and the like. Such an antibody can be obtained from a serum after animals were immunized with the HD/TF-1 and/or the HD/TF-2 described above or a peptides derived from them as antigens. Alternatively, such an antibody can be prepared by introducing the eukaryotic expression vector described above into the muscle or the skin of animals by injection or gene gun, and then by collecting a serum. As animals, mouse, rat, rabbit, goat, chicken and the like can be used. A monoclonal antibody can be obtained when myeloma cells are fused with B cells from the spleen of immunized animal, and hybridoma is made.

The invention is further detailed in the following Examples, which are not intended to restrict the invention.

### EXAMPLES

### Example 1

### Isolation of the HD Gene Transcriptional Factor

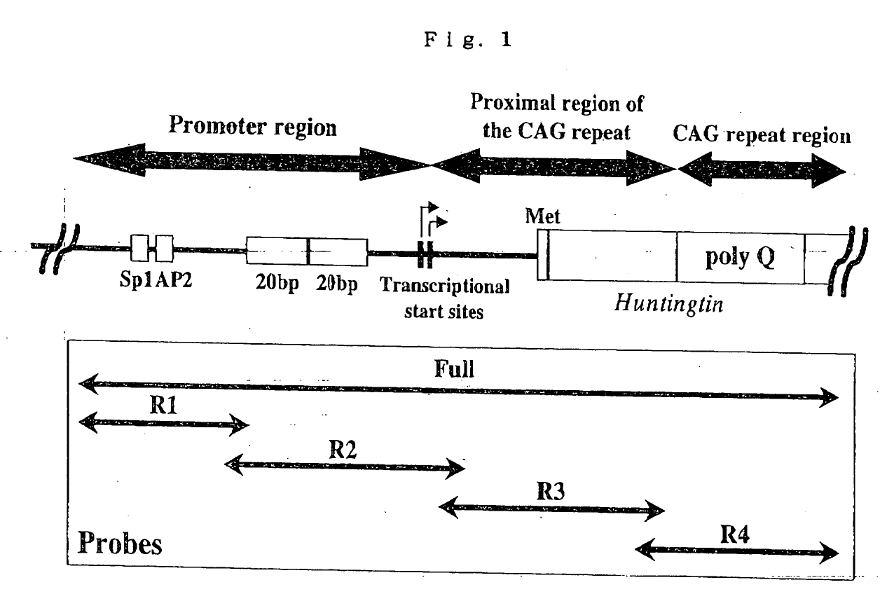

In order to isolate a transcriptional factor of the HD gene, the region from −364 to +158 in the HD gene (including promoter region, transcription initiation point, CAG repeat sequence: see FIG. 1) was amplified by PCR, and inserted into the SmaI site of a pHISi or pHISi-1 to generate a reporter construct. By means of yeast One-Hybrid system with this reporter construct, a human testicular cDNA library (CLONTECH, Human Testis MATCHMAKER cDNA library) was screened. As a result, three positive cDNA clones 2, 8 and 11 were identified.

All procedures of the One-Hybrid system were in accordance with the CLONTECH MATCHMAKER One-Hybrid system Protocol.

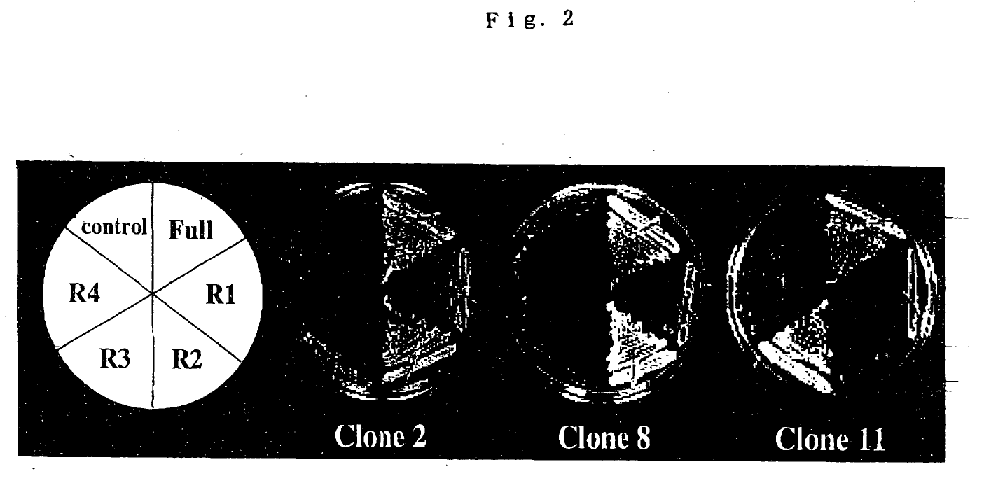

Subsequently, to refine the binding region of each clone, the region from −364 to +158 in the HD gene was further divided into the following 4 regions, i.e., −364 to −213 (R1), -230 to −113 (R2), -131 to +51 (R3) and +29 to +158 (R4), and these reporter constructs produced (see FIG. 1) were subjected to the analysis using the yeast One-Hybrid system. As shown in FIG. 2, it was proved that clones 2 and 8 among 3 positive clones showed the binding activity to the R2 region which is the HD gene transcription regulatory region. Thus, it was demonstrated that the expression products of the cDNA clone 2 (about 1.3 kb) and the cDNA clone 8 (about 1.8 kb) were the HD gene transcriptional factors.

The cDNA clones 2 and 8 were found to be inserted into the EcoRI-XhoI site of the vector pACT2, respectively.

### Example 2

### Clone 2 and Clone 8 Expression Analysis

The expression of the cDNA clone 2 and the cDNA clone 8, which were isolated in Example 1, in human tissues was analyzed by Northern blotting. Northern blotting was conducted by using a TOYOBO Gene Hynter™ membrane (Human Normal Tissue mRNA blot I, II, III, IV). The EcoRI-XhoI site fragment of the pACT2 containing the cDNA clones 2 or 8 was labeled with a radioisotope and used as a probe DNA. In 1.1× Hybrid solution (0.55M sodium phosphate-1.1 mM EDTA-7.7% SDS) containing a Herring testis DNA at 10 mg/ml, a membrane and the probe DNA were hybridized at 65° C. overnight. The membrane was washed twice with 2×SSC at room temperature for 5 minutes, followed by once with 2×SSC-1% SDS at 65° C. for 30 minutes. Thereafter, the membrane was exposed to an X-ray film to detect signals.

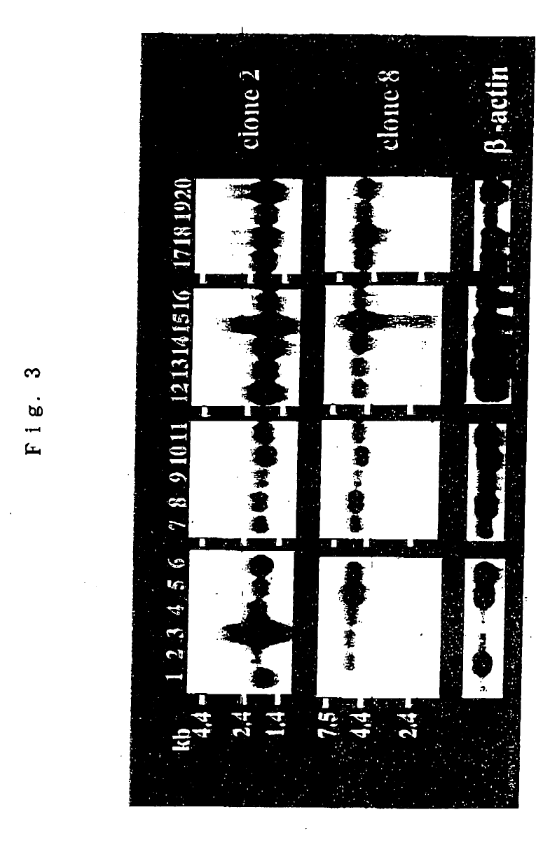

As a result, it was proved that a human gene corresponding to each of the cDNA clones 2 and 8 showed ubiquitous expression pattern as shown in FIG. 3. It was also proved that the mRNA size of the cDNA clone 2 was about 1.8 kb and the mRNA size of the cDNA clone 8 was about 5.5 kb.

### Example 3

### Acquisition of Full-Length cDNA

Since the cDNA clones 2 and 8 obtained in Example 1 were smaller than the mRNA sizes from the results of Example 2, the respective full-length cDNAs were isolated by the method described below.

Thus, about the clone 2, a cDNA library was prepared using the human testis-derived mRNA (CLONTECH, Human Testis mRNA), and was screened using a probe that the EcoRI-XhoI site fragment of the pACT2 containing the cDNA clones 2 was labeled with a radioisotope. The cDNA library was prepared by using the STRATAGENE ZAP-cDNA synthesis kit in accordance with the protocol thereof.

As a result, two cDNA carrying different sizes were isolated. One has ˜1.5 kb DNA size (cDNA clone 2M) and the other has 1.8 kb DNA size (cDNA clone 2L), thus it was demonstrated that the cDNA clone 2L was the full-length cDNA. The expression product of the cDNA clone 2L was designated as the HD gene transcriptional factor HD/TF-1.

On the other hand, about the clone 8, although a full-length cDNA was not isolated by the screening of the cDNA library, a cDNA containing the 5′ region was obtained by a 5′ RACE (rapid amplification of cDNA ends). The template mRNA used was a CLONTECH Human Testis mRNA, and PCR primers used were synthetic oligonucleotides represented by SEQ ID No. 5 (the region from 368 nt to 393 nt of cDNA clone 8) and SEQ ID No. 6 (the region from 312 nt to 333 nt of cDNA clone 8). This 5′ RACE was conducted by using the CLONTECH SMART™ RACE cDNA Amplification Kit in accordance with the protocol thereof.

As a result, it was proved that the 5′ region fragment obtained in Example 1 have already encoded the translation region containing the initiation codon (methionine) completely. It was judged that the difference between the cDNA size and the mRNA size of the clone 8 was attributable to the difference in the length of the 3′ non-translation region. The protein encoded by the cDNA clone 8 was designated as the HD gene transcriptional factor HD/TF-2.

### Example 4

### Recombinant Expression Vector Construction

Each of the translation region of the cDNA clone 2L (amino acid positions corresponding to 1-387 of SEQ ID No. 2) and the translation region of the cDNA clone 8 (amino acid positions corresponding to 1-513 of SEQ ID No. 4) was amplified by PCR, and inserted into the EcoRI-SalI site of the expression vector pEGFP-N and pEGFP-C, the EcoRI-XhoI site of the expression vectors pGEX-5×, pcDNA3.1/Myc-His(+) and pcDNA3.1/His, and the NheI-NstI site (cDNA clone 2L) and the NheI site (cDNA clone 8) of the expression vector pBsCAG-2 to generate the recombinant expression vectors, respectively. The DNA fragments of the cDNA clone 2L corresponding to each of the amino acid positions, 1-105, 106-321 and 322-387 and the DNA fragments of the cDNA clone 8 corresponding to each of the amino acid positions, 1-168, 169-437 and 438-513, were amplified by PCR, and inserted into the EcoRI-XhoI site of the expression vector pGEX-5 to generate the recombinant expression vectors, respectively. The constructed recombinant vectors are as follows.
Vector 1: pEGFP-N/2L (1-387)
Vector 2: pEGFP-N/8 (1-513)
Vector 3: pEGFP-C/2L (1-387)
Vector 4: pEGFP-C/8 (1-513)
Vector 5: pGEX-5X/2L (1-387)
Vector 6: pGEX-5X/8 (1-513)
Vector 7: pcDNA3.1/Myc-His(+)/2L (1-387)
Vector 8: pcDNA3.1/Myc-His(+)₈ (1-513)
Vector 9: pcDNA3.1/His/2L (1-387)
Vector 10: pcDNA3.1/His/8 (1-513)
Vector 11: pBsCAG-2/2L (1-387)
Vector 12: pBsCAG-2/8 (1-513)
Vector 13: pGEX-5/2L (1-105)
Vector 14: pGEX-5/2L (106-321)
Vector 15: pGEX-5/2L (322-387)
Vector 16: pGEX-5/8 (1-168)
Vector 17: pGEX-5/8 (169-437)
Vector 18: pGEX-5/8 (438-513)

### Example 5

### In Vitro DNA Binding Assay

Whether each gene product of clone 2L and clone 8 obtained in Example 3 has the DNA-binding ability in vitro was analyzed by a gel shift assay. The DNA fragments used in Example 1, the R1, R2, R3 and R4, were labeled with a radioisotope, and used as probes. The target proteins were a GST-fusion protein of the clone 2L gene product expressed by the vector 5 (HD/TF-1) and a GST-fusion protein of the clone 8 gene product expressed by the vector 6 (HD/TF-2) constructed in Example 4.

Thus, the incubation was performed in a reaction solution (20 mM Tris-HCl (pH7.6)-50 mM KCl-10% Glycerol-1 mM DDT) containing 50 ng to 200 ng of each fusion protein and 2 μg of poly(dI-dC) at room temperature for 5 minutes, and then 0.1 ng of the ³²P-labeled probe DNA was added into the reaction solution and allowed to undergo the binding reaction at room temperature for 20 minutes. After completion of the reaction, the reaction solution was subjected to 4% PAGE (polyacrylamide gel electrophoresis). After the electrophoresis, the gel was dried, and exposed to the X-ray film to detect signals.

The competitive activity test was carried out using 100 ng of the fusion proteins expressed by the vectors 5 and 6 generated in Example 4 and performed under the same condition of the binding activity test as described above, except for the conditions described below. Thus, 10 ng (100-fold amount of probe DNA) of a non-labeled DNA was added as a competitor DNA during the incubation at room temperature for 5 minutes, and then 0.1 ng of the ³²P-labeled probe DNA was added into the reaction solution, and allowed to undergo the binding reaction at room temperature for 20 minutes. The probe DNAs used were the R2 and R3 fragments.

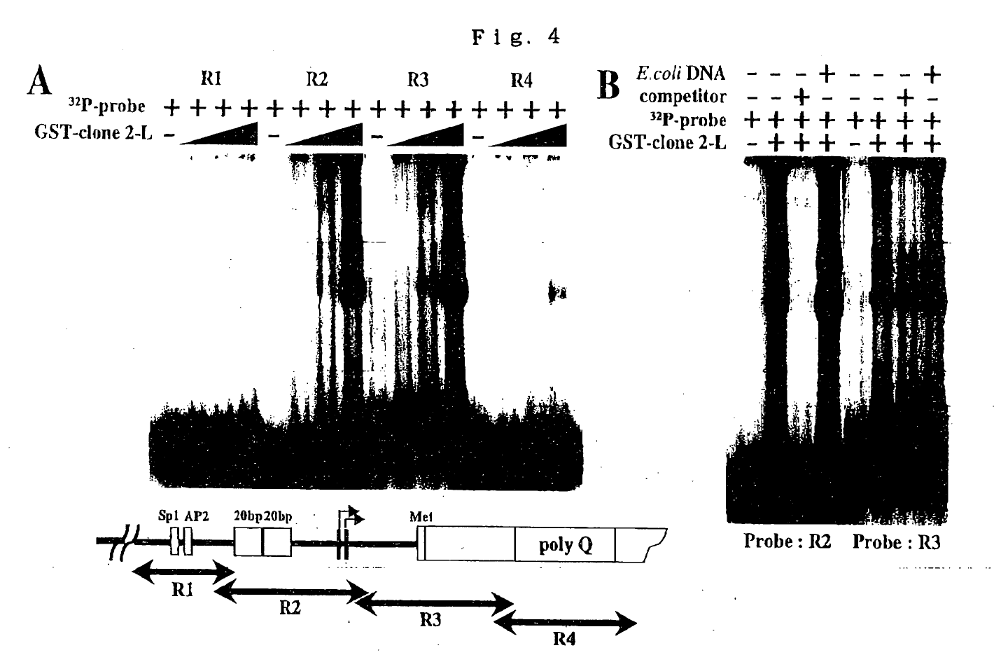

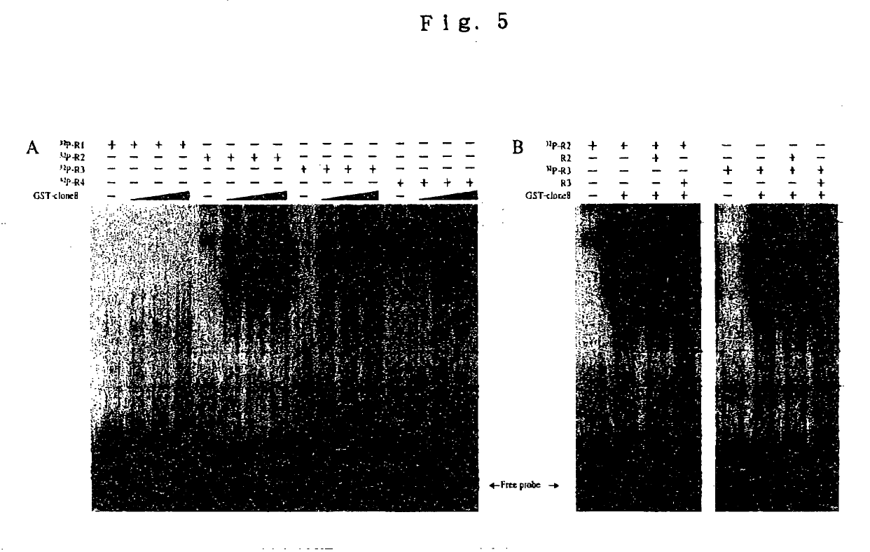

The results of the binding activity test are shown in FIG. 4A and FIG. 5A, which indicated that the GST-fusion protein of the clone 2L gene product (HD/TF-1) and that of the clone 8 gene product (HD/TF-2) showed the binding activity to the labeled R2 probe and R3 probe.

The results of the competitive activity test are shown in FIG. 4B and FIG. 5B, which demonstrated that the binding of the fusion proteins was specific to the R2 region, but non-specific to the R3 region.

### Example 6

### Domain Function Analysis

From the results of Example 5, it was proved that the clone 2L gene product (HD/TF-1) and the clone 8 gene product (HD/TF-2) specifically bound to the R2 region of the HD gene. Next, an analysis was conducted to know which region in the clone 2L gene product interacts with the HD gene.

Thus, the fusion proteins expressed by each of the vectors 13, 14 and 15 generated in Example 4 were used for the binding activity test as describe in Example 5. As a probe DNA, the labeled R2 fragment was used.

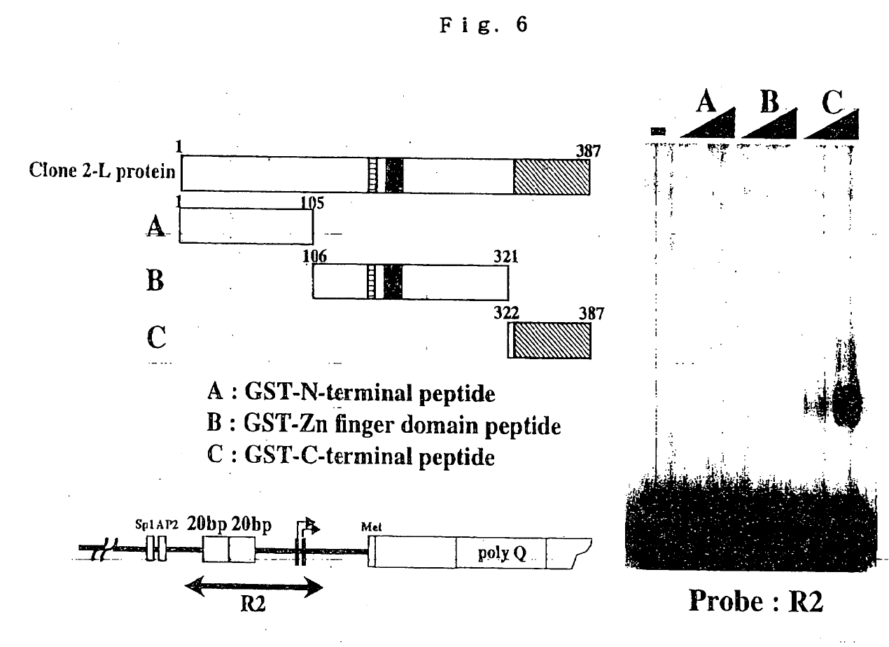

The results are shown in FIG. 6. It indicated that the C-terminal of the clone 2L gene product showed the binding activity to the R2 probe DNA, revealing that this region was the binding region to the HD gene transcriptional regulatory region.

Subsequently, an analysis was conducted to investigate which sequence in the HD gene transcriptional regulatory region is recognized by this C-terminal region. Ten ng to 400 ng of each fusion protein expressed by the vector 15 and the vector 18, which encoded the C-terminal region of the clone 8 gene product exhibiting a high homology with the C-terminal region of the clone 2L gene product, constructed in Example 4 was used and subjected to the binding test as describe in Example 5. The probe DNA used was the labeled R1+R2 fragment (−364 to −113). Subsequently, each of these reaction solutions was combined with 1/10 volume of 10× DNase I solution (15 ug/ml DNaseI, 50 mM MgCl₂) and subjected to the reaction of the enzyme digestion for 1 minutes at 25° C. The reaction was terminated by the addition of DNase I stop solution (1.6M ammonium acetate, 95 mM EDTA, 0.8% SDS, 0.3 mg/ml Calf thymus DNA), and the DNA fragments were purified. The purified DNA was dissolved in a loading dye (80% formamide, 10 mM EDTA (pH8), 0.025% bromophenol blue, 0.025% xylene cyanol), and separated by an electrophoresis using a 8M urea-8% acrylamide gel.

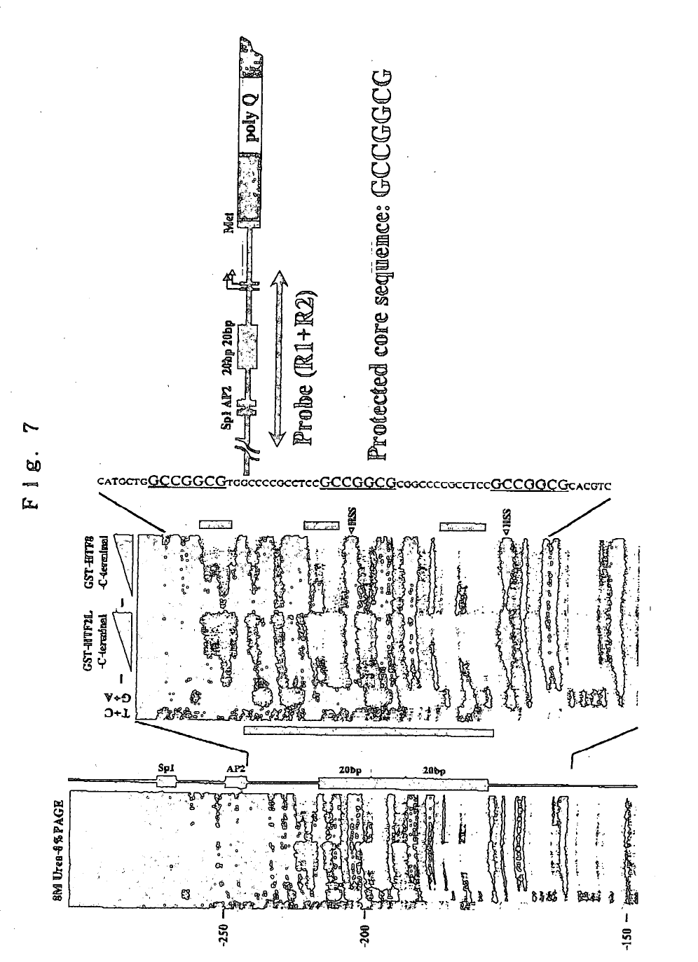

As a result, as shown in FIG. 7, since the C-terminal regions (DNA binding domain) of the clone 2L and clone 8 gene products recognized the 7 bp gccggcg sequence in the HD gene transcriptional regulatory region, and also since this sequence locates in the three sites at intervals of 13 bp spacer sequences, it was proved that these regions constituted a novel cis-element in the HD gene transcriptional regulatory regions.

### Example 7

### Intracellular Local Analysis

The vectors 1 to 4 generated in Example 4 were transfected into HeLa cells. After 24 hours, cells were fixed in 4% paraformaldehyde, and the fluorescent was observed under microscope.

As a result, the fusion protein of the clone 2L gene product with GFP partially showed nuclear localization, but the expression of the fusion protein of clone 8 gene product was observed only in the cytoplasm.

### INDUSTRIAL APPLICABILITY

As detailed above, the present invention provides the HD gene transcriptional factor. This transcriptional factor enables a development of a therapeutic technology for an artificial control of a pathogenically mutated huntingtin production as well as the development of a therapeutic method or agent against a selective neuronal cell death in HD.

1. An isolated and purified transcriptional factor for the Huntington's disease gene, which has the amino acid sequence of SEQ ID No. 2.

2. The transcriptional factor for the Huntington's disease gene of claim 1, which recognizes at least one of the 144th to 150th sequence, the 164th to 170th sequence and the 184th to 190th sequence in the base sequence of SEQ ID No. 7 that is present in transcriptional regulatory region of Huntington's disease gene.

3. An isolated and purified transcriptional factor for the Huntington's disease gene, which has the amino acid sequence of SEQ ID No. 4.

4. The transcriptional factor for the Huntington's disease gene of claim 3, which recognizes at least one of the 144th to 150th sequence, the 164th to 170th sequence and the 184th to 190th sequence in the base sequence of SEQ ID No. 7 that is present in transcriptional regulatory region of Huntington's disease gene.

5. An isolated and purified polynucleotide encoding the transcriptional factor for the Huntington's disease gene of claim 1.

6. The polynucleotide of claim 5 having the base sequence of SEQ ID No. 1.

7. An isolated and purified polynucleotide encoding the transcriptional factor for the Huntington's disease gene of claim 3.

8. The polynucleotide of claim 7 having the base sequence of SEQ ID No. 3.

9. An oligonucleotide consisting of a base sequence of 10 base pairs or more which hybridizes with the polynucleotide of claim 5.

10. An oligonucleotide consisting of a base sequence of 10 base pairs or more which hybridizes with the polynucleotide of claim 7.

11. A recombinant vector holding the polynucleotide of claim 5.

12. A recombinant vector holding the polynucleotide of claim 7.

13. Transformed cells with the recombinant vector of claim 11.

14. Transformed cells with the recombinant vector of claim 12.

15. An antibody against the transcriptional factor for the Huntington's disease gene of claim 1.

16. An antibody against the transcriptional factor for the Huntington's disease gene of claim 3.

17. An oligonucleotide consisting of a base sequence of 10 base pairs or more which hybridizes with the polynucleotide of claim 6.

18. An oligonucleotide consisting of a base sequence of 10 base pairs or more which hybridizes with the polynucleotide of claim 8.

19. A recombinant vector holding the polynucleotide of claim 6.

20. A recombinant vector holding the polynucleotide of claim 8.

21. Transformed cells with the recombinant vector of claim 19.

22. Transformed cells with the recombinant vector of claim 20.
